(19) United States
(12) Patent Application Publication
(10) Pub. No.: US 2009/0281126 A1
(43) Pub. Date: Nov. 12, 2009

(54) Organic Compounds

(75) Inventors: Robin Alec Fairhurst, Horsham (GB); Roger John Taylor, Horsham (GB)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/296,714

(22) PCT Filed: Apr. 19, 2007
(86) PCT No.: PCT/EP07/53847

§ 371 Date: Oct. 10, 2008

(30) Foreign Application Priority Data
Apr. 21, 2006 (GB) 0607944.6

(51) Int. Cl. A61K 31/52; C07D 473/34; A61P 19/02

(52) U.S. Cl. 514/263.2; 544/277; 514/263.4

(57) ABSTRACT

A compound of formula (I) and their preparation and use as pharmaceuticals

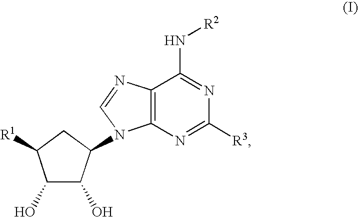

wherein R¹, R² and R³ are as defined herein.

This invention relates to organic compounds, their preparation and use as pharmaceuticals.

In one aspect, the present invention provides compounds of formula (I)

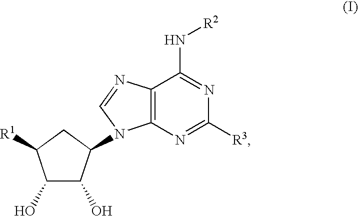

in free or salt form, wherein
R¹ denotes a N-bonded 3- to 12-membered heterocyclic group containing from 1 to 4 ring nitrogen atoms and optionally containing from 1 to 4 other heteroatoms selected from the group consisting of oxygen and sulfur, that group being optionally substituted by oxo, C₁-C₈-alkoxy, C₆-C₁₀-aryl, R^{1a} or by C₁-C₈-alkyl optionally substituted by OH, or
R¹ is —NH—C₁-C₈-alkylcarbonyl optionally substituted by OH, —NH—C₃-C₈-cycloalkylcarbonyl, —NH—SO₂—C₁-C₈-alkyl, —NH—C₇-C₁₄-aralkylcarbonyl, —NH—C(═O)-3- to 12-membered heterocyclic group, —NH—C(═O)—C₆-C₁₀-aryl or —NH—C(═O)—C(═O)—NH—C₁-C₈-alkyl optionally substituted by R^{1a}, where R^{1a} is a 3- to 12-membered heterocyclic group containing at least one ring heteroatom selected from the group consisting of nitrogen, oxygen and sulphur, said 3- to 12-membered heterocyclic ring being optionally substituted by halo, cyano, oxo, OH, carboxy, amino, nitro, C₁-C₈-alkyl, C₁-C₈-alkylsulfonyl, aminocarbonyl, C₁-C₈-alkylcarbonyl or C₁-C₈-alkoxy optionally substituted by aminocarbonyl;
R² is selected from the group consisting of C₁-C₈-alkyl, R— and S— 1-phenylethyl, an unsubstituted benzyl group, and a phenylethyl or benzyl group substituted in one or more positions with a substituent selected from the group consisting of C₁-C₈-alkyl, amino, halo, C₁-C₈-haloalkyl, nitro, OH, acetamido, C₁-C₈-alkoxy and sulfo, or
R² is

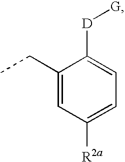

where
   R^{2a} is halo, trifluoromethyl, cyano, C₁-C₈-alkyl, C₁-C₈-alkyloxy, ethenyl or ethynyl;
   D is oxy, thio, NH, C₁-C₈-alkyloxy, C₁-C₈-alkylthio or —CO-alkylamino; and
   G is a partially saturated, fully saturated or fully unsaturated 5- to 8-membered ring optionally having 1 to 3 heteroatoms selected independently from oxygen, sulfur and nitrogen, or, a bicyclic ring consisting of two fused partially saturated, fully saturated or fully unsaturated 3- to 6-membered rings, taken independently, optionally having 1 to 4 heteroatoms selected independently from nitrogen, sulfur and oxygen; wherein said G is optionally mono-, di- or tri-substituted independently with halo, C₁-C₈-alkyl, trifluoromethyl, trifluoromethoxy, nitro, cyano, C₃-C₁₀-cycloalkyl, hydroxy or C₁-C₈-alkoxy, or
   G is cyano, C₁-C₈-alkoxycarbonyl, C₃-C₁₀-cycloalkoxycarbonyl, C(O)NR⁴R⁵, C(S)NR⁴R⁵, C(NH)NR⁴NR⁵, C(N(C₁-C₃)alkyl)NR⁴R⁵ or C(N(C₃-C₁₀)cycloalkyl)NR⁴R⁵;
R³ is selected from H, halo, C₁-C₈-alkyl optionally substituted by halo or OH, C₁-C₈-alkoxy, amino, C₁-C₈-alkylamino, C₂-C₁₀-alkenes, C₂-C₁₀-alkynes optionally substituted by C₁-C₈-alkyl, aryl optionally substituted by C₁-C₈-alkyl or OH, thio and C₁-C₈-alkylthio;
   R⁴ is a bond, H, C₁-C₁₀-alkyl, hydroxy, C₁-C₁₀-alkoxy, C₃-C₁₀-cycloalkoxy or a partially saturated, fully saturated or fully unsaturated 5- to 8-membered ring, optionally linked through C₁-C₈-alkyl, optionally having 1 to 3 heteroatoms selected independently from oxygen, sulfur and nitrogen, or, a bicyclic ring or a bicyclic ring with optional C₁-C₈-bridge optionally linked through C₁-C₈-alkyl, said bicyclic ring or bridged bicyclic ring optionally having 1 to 4 heteroatoms selected independently from nitrogen, sulfur and oxygen wherein said C₁-C₁₀-alkyl, C₁-C₁₀-alkoxy, C₃-C₁₀-cycloalkoxy or R⁴ ring(s) is optionally mono-, di- or tri-substituted independently with halo, C₁-C₈-alkyl, trifluoromethyl, nitro, cyano, C₃-C₁₀-cycloalkyl, OH or C₁-C₈-alkoxy;
   R⁵ is a bond, H, C₁-C₈-alkyl or C₁-C₁₀-cycloalkyl, or
   R⁴ and R⁵, taken together with the nitrogen to which they are attached, form a fully saturated or partially unsaturated 4- to 9-membered ring, said ring optionally bridged, optionally having 1 to 3 heteroatoms selected independently from oxygen, sulfur and nitrogen, said ring optionally mono- or di-substituted independently with oxo, hydroxy, C₁-C₈-alkoxy, C₁-C₈-alkyl, amino, mono —N— or di-N,N—C₁-C₈-alkylaminocarbonyl, mono-N— or di-N,N—C₃-C₁₀-cycloalkylaminocarbonyl, N—C₁-C₈-alkyl-N—C₃-C₁₀-cycloalkylaminocarbonyl, mono-N— or di-N,N—C₁-C₈-alkylamino, mono —N— or di-N,N—C₃-C₁₀-cycloalkylamin, N—C₁-C₈-alkyl-N—C₃-C₁₀-cycloalkylamino, formylamino, C₁-C₈-alkylcarbonylamino, C₃-C₁₀-cycloalkylcarbonylamino, C₁-C₈-alkoxycarbonylamino, N—C₁-C₈-alkoxycarbonyl —N—C₁-C₈-alkylamino, C₁-C₈-sulfamoyl, C₁-C₈-alkylsulfonylamino, C₃-C₁₀-cycloalkylsulfonylamino or a partially saturated, fully saturated or fully unsaturated 5- to 8-membered ring, optionally linked through C₁-C₈-alkyl, optionally having 1 to 3 heteroatoms selected independently from oxygen, sulfur and nitrogen, or, a bicyclic ring consisting of two fused partially saturated, fully saturated or fully unsaturated 3- to 6-membered rings, taken independently, optionally linked through C₁-C₈-alkyl, optionally having 1 to 4 heteroatoms selected independently from nitrogen, sulfur and oxygen, and optionally mono- or di-substituted with halo, trifluoromethyl, trifluoromethoxy, C₁-C₈-alkyl or C₁-C₈-alkoxy.

In another aspect, the present invention provides compounds of formula (I)

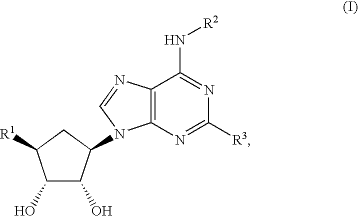

in free or salt form, wherein
R¹ denotes a N-bonded 3- to 12-membered heterocyclic group containing from 1 to 4 ring nitrogen atoms and optionally containing from 1 to 4 other heteroatoms selected from the group consisting of oxygen and sulphur, or
R¹ is —NH—C₁-C₈-alkylcarbonyl;
R² is C₁-C₈-alkyl or benzyl optionally substituted by halogen, or
R² is

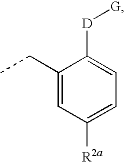

where
   R^{2a} is halo, trifluoromethyl, cyano, C₁-C₈-alkyl, C₁-C₈-alkoxy, ethenyl or ethynyl;
   D is oxy, thio, NH, C₁-C₈-alkoxy, C₁-C₈-alkylthio or —CO-alkylamino; and
   G is a partially saturated, fully saturated or fully unsaturated 5- to 8-membered ring optionally having 1 to 3 heteroatoms selected independently from oxygen, sulfur and nitrogen, or, a bicyclic ring consisting of two fused partially saturated, fully saturated or fully unsaturated 3- to 6-membered rings, taken independently, optionally having 1 to 4 heteroatoms selected independently from nitrogen, sulfur and oxygen; wherein said G is optionally mono-, di- or tri-substituted independently with halo, C₁-C₈-alkyl; and
R³ is selected from H, halo, C₁-C₈-alkyl optionally substituted by halo or OH, C₁-C₈-alkoxy, amino, C₁-C₈-alkylamino, C₂-C₁₀-alkene s, C₂-C₁₀-alkynes optionally substituted by C₁-C₈-alkyl, C₆-C₁₀-aryl optionally substituted by C₁-C₈-alkyl or OH, thio and C₁-C₈alkylthio.

According to formula (I), R¹ is suitably a 5- to 12-membered heterocyclic group containing at least one ring heteroatom selected from the group consisting of nitrogen, oxygen and sulphur. Preferably R¹ is a 5- to 6-membered heterocyclic group, such as a triazole.

According to formula (I), R¹ is also suitably —NH—C₁-C₈-alkylcarbonyl. The —NH—C₁-C₈-alkylcarbonyl is preferably —NHC(O)CH₃.

According to formula (I), R² is suitably

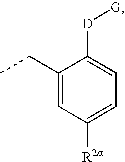

where
R^{2a} is suitably a halogen, such as chlorine;
D is suitably C₁-C₈-alkoxy; and
G is suitably 5-membered heterocyclic group, such as isoxazole mono-substituted by a methyl group.

According to formula (I), R² is also suitably a benzyl group mono-substituted by halogen. Preferably the halogen is iodine.

According to formula (I), R² is also suitably C₁-C₈-alkyl. Preferably methyl.

According to formula (I), R³ is suitably H, halo or C₂-C₁₀-alkynes optionally substituted by C₁-C₈-alkyl.

### DEFINITIONS

Terms used in the specification have the following meanings:

“Optionally substituted” means the group referred to can be substituted at one or more positions by any one or any combination of the radicals listed thereafter.

“Halo” or “halogen”, as used herein, may be fluorine, chlorine, bromine or iodine.

“Hydroxy”, as used herein, is OH.

“C₁-C₈-alkyl”, as used herein, denotes straight chain or branched alkyl having 1 to 8 carbon atoms. Preferably C₁-C₈-alkyl is C₁-C₄-alkyl.

“C₁-C₈-alkoxy”, or as used herein, denotes straight chain or branched alkoxy having 1 to 8 carbon atoms, e.g., O—C₁-C₈-alkyl. Preferably, C₁-C₈-alkoxy is C₁-C₄-alkoxy.

“C₁-C₈-alkylamino” and “di-C₁-C₈-alkyl-amino”, as used herein, denote amino substituted respectively by one or two C₁-C₈-alkyl groups as hereinbefore defined, which may be the same or different.

“C₁-C₈-alkylcarbonyl” and “C₁-C₈-alkoxycarbonyl”, as used herein, denote C₁-C₈-alkyl or C₁-C₈-alkoxy, respectively, as hereinbefore defined attached by a carbon atom to a carbonyl group.

“C₆-C₁₀-aryl”, as used herein, denotes a monovalent carbocyclic aromatic group that contains 6 to 10 carbon atoms and which may be, e.g., a monocyclic group, such as phenyl; or a bicyclic group, such as naphthyl.

“C₇-C₁₄-aralkyl”, as used herein, denotes alkyl, e.g., C₁-C₄-alkyl, as hereinbefore defined, substituted by C₆-C₁₀-aryl as hereinbefore defined. Preferably, C₇-C₁₄-aralkyl is C₇-C₁₀-aralkyl, such as phenyl-C₁-C₄-alkyl.

“C₁-C₈-alkylaminocarbonyl” and “C₃-C₈-cycloalkylaminocarbonyl” as used herein denote C₁-C₈-alkylamino and C₃-C₈-cycloalkylamino respectively as hereinbefore defined attached by a carbon atom to a carbonyl group. Preferably C₁-C₈-alkylaminocarbonyl and C₃-C₈-cycloalkyl-aminocarbonyl are C₁-C₄-alkylaminocarbonyl and C₃-C₈-cycloalkylaminocarbonyl, respectively.

“C₃-C₁₅-carbocyclic group”, as used herein, denotes a carbocyclic group having 3 to 15 ring carbon atoms, e.g., a monocyclic group, either aromatic or non-aromatic, such as a cyclopropyl, cyclopentyl, cyclohexyl, cycloheptyl, cyclooctyl or phenyl; or a bicyclic group, such as bicyclooctyl, bicyclononyl, bicyclodecyl, indanyl or indenyl, again any of which can be substituted by one or more, usually one or two, C₁-C₄-alkyl groups.

“3- to 12-membered heterocyclic ring containing at least one ring heteroatom selected from the group consisting of nitrogen, oxygen and sulfur”, as used herein, may be, e.g., furan, pyrrole, pyrrolidine, pyrazole, imidazole, triazole, isotriazole, tetrazole, thiadiazole, isothiazole, oxadiazole, pyridine, piperidine, pyrazine, oxazole, isoxazole, pyrazine, pyridazine, pyrimidine, piperazine, pyrrolidine, morpholino, triazine, oxazine or thiazole. Preferred heterocyclic rings include piperazine, pyrrolidine, morpholino, imidazole, isotriazole, pyrazole, tetrazole, thiazole, triazole, thiadiazole, pyridine, piperidine, pyrazine, furan, oxazole, isoxazole, oxadiazole and azetidine. The 3-to-12-membered heterocyclic ring can be unsubstituted or substituted.

Throughout this specification and in the claims that follow, unless the context requires otherwise, the word “comprise”, or variations, such as “comprises” or “comprising”, will be understood to imply the inclusion of a stated integer or step or group of integers or steps but not the exclusion of any other integer or step or group of integers or steps. As understood by one skilled in the art only combinations of substituents that are chemically possible are embodiments of the invention.

Especially preferred specific compounds of formula (I) are those described hereinafter in the Examples.

Stereoisomers are those compounds where there is an asymmetric carbon atom. The compounds exist in individual optically active isomeric forms or as mixtures thereof, e.g., as diastereomeric mixtures. The present invention embraces both individual optically active R and S isomers, as well as mixtures thereof. Individual isomers can be separated by methods well known to those skilled in the art, e.g. chiral high performance liquid chromatography (HPLC).

Tautomers are one of two or more structural isomers that exist in equilibrium and are readily converted from one isomeric form to another.

The compounds of the invention may exist in both unsolvated and solvated forms. The term “solvate” is used herein to describe a molecular complex comprising the compound of the invention and one or more pharmaceutically acceptable solvent molecules, e.g., ethanol. The term “hydrate” is employed when said solvent is water.

### Synthesis

The Invention also provides, in another aspect, a method of preparing a compound of formula (I), in free or salt form which comprises:
(i) (A) for the preparation of compounds of formula (I), reacting a compound of formula (Ia)

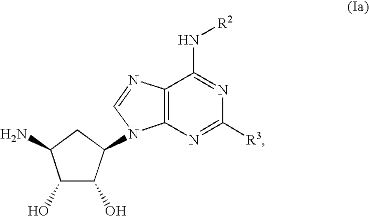

where R² and R³ are as hereinbefore defined, with acetyl chloride in the presence of base;
(B) for the preparation of compounds of formula (I), where R³ is C₂-C₈-alkynyl, reacting a compound of formula (Ib)

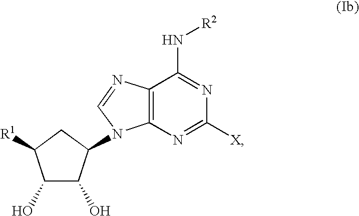

where X is a leaving group, with a compound of formula

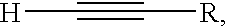

where R can be C₁-C₆-alkyl;
(C) for the preparation of compounds of formula (I), reacting a compound of formula (Ic)

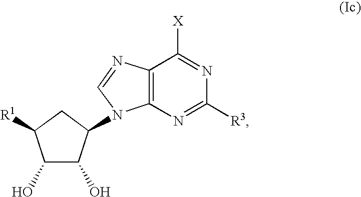

where
   R¹ and R³ are as hereinbefore defined; and
   X is a leaving group, with a compound of formula H₂N—R², where R² is as hereinbefore defined in the presence of a base; and
(ii) recovering the resultant compound of formula (I), in free or pharmaceutically acceptable salt form.

The compounds of formula (I) can be prepared, e.g., using the reactions and techniques described below and in the Examples. The reactions may be performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of formula (I) into another compound of formula (I). Examples of such manipulations are conversion of an ester or a ketone to an alcohol; conversion of an ester to a ketone; interconversions of esters, acids and amides; alkylation, acylation and sulfonylation of alcohols and amines; and many others. Substituents can also be added using common reactions, such as alkylation, acylation, halogenation or oxidation. Such manipulations are well-known in the art, and many reference works summarize procedures and methods for such manipulations. Some reference works which gives examples and references to the primary literature of organic synthesis for many functional group manipulations, as well as other transformations commonly used in the art of organic synthesis are March's Organic Chemistry, 5^{th} Edition, Wiley and Chichester, Eds. (2001); Comprehensive Organic Transformations, Larock, Ed., VCH (1989); Comprehensive Organic Functional Group Transformations, Katritzky et al. (series editors), Pergamon (1995); and Comprehensive Organic Synthesis, Trost and Fleming (series editors), Pergamon (1991). It will also be recognized that another major consideration in the planning of any synthetic route in this field is the judicious choice of the protecting group used for protection of the reactive functional groups present in the compounds described in this invention. Multiple protecting groups within the same molecule can be chosen such that each of these protecting groups can either be removed without removal of other protecting groups in the same molecule, or several protecting groups can be removed using the same reaction step, depending upon the outcome desired. An authoritative account describing many alternatives to the trained practitioner is Protective Groups In Organic Synthesis, Greene and Wuts, Eds., Wiley and Sons (1999). It is understood by those skilled in the art that only combinations of substituents that are chemically possible are embodiments of the present invention.

### Pharmacological Activity and Use

Compounds of formula (I) and their pharmaceutically acceptable salts are useful as pharmaceuticals. In particular, they activate the adenosine A3 receptor, i.e., they act as A2A receptor agonists. Their properties as A3 agonists are described in WO 05/063246, WO 02/055085, WO 95/02604 and WO 06/011130.

Compounds of the Examples hereinbelow have Ki values and EC₅₀ values below 5.0 μM in the following assays. For example, the compound of Example 1 has a Ki value of 0.91 nM in the Ki binding assay and a EC₅₀ value of 11.0 nM in the A₃ [³⁵S]-GTP_{Gamma}S functional assay.

### A3 Binding Assay Protocol

### List of Abbreviations

| | |
|---|---|
| A₃ | Adenosine A₃ receptor |
| BSA | Bovine serum albumin |
| CHO | Chinese hamster ovary |
| DMSO | Dimethyl sulphoxide |
| EDTA | Ehylenediaminetetraacetic acid |
| FCS | Fetal calf serum |
| HEPES | 4-(2-Hydroxyethyl)piperazine-1-ethanesulfonic acid |
| I-AB-MECA | N6-(4-Amino-3-iodobenzyl)-5′-N-methylcarbamoyl- |
| | adenosine |
| K_{d} | Dissociation constant |
| MgCl₂ | Magnesium chloride |
| NaCl | Sodium chloride |
| Tris-HCl | Tris(hydroxymethyl)-aminomethane hydrochloride |

### Introduction

Adenosine, an endogenous modulator of a wide range of biological functions, interacts with at least four cell surface receptor subtypes classified as A₁, A_{2A}, A_{2B} and A₃, all of which are coupled to G proteins. See Linden, Annu Rev Pharmacol Toxicol, Vol. 41, pp. :775-787 (2001).

Until recently, most of the anti-inflammatory actions of adenosine were thought to be produced through A_{2A} receptors. However, the A₃ subtype, may play a basic role in different pathologies such as inflammation and neurodegeneration [see Kohno et al., Biochem Biophys Res Commun, Vol. 219, pp. 904-910 (1996)] and asthma [see Jacobson et al., Neuropharmacology, Vol. 36, pp. 1157-1165 (1997)].

The adenosine derivative, 4-aminobenzyl-5′-N-methyl-carboxamidoadenosine (AB-MECA), is a potent A₃ receptor selective agonist which is used as a reference compound. See Varani et al., Life Sci, Vol. 63, No. 5, pp. 81-87 (1998).

Compounds in the present invention were tested in an A₃ binding assay using the iodinated ligand [¹²⁵I]-AB-MECA with membranes prepared from CHO cells stably expressing human A₃ receptors.

### Methods

### Materials

CHO adenosine A3 membranes
[¹²⁵I]-AB-MECA: Amersham Pharmacia Biotech (Cat# TRK)
CGS21680: Tocris (1063)
Unifilter GF/B 96-well plates: Perkin Elmer (Cat# 6005174)
96-well U bottom polypropylene plates: Greiner (Cat# 650201)
TopSeal: Canberra Packard (Cat# 6005185)
BSA: Sigma Cat# A-6003
Adenosine deaminase (1000 U/mL): Roche Diagnostics Limited (Cat# 102121)
Microscint-20 (1 L): Perkin Elmer (Cat# 6013611)
All other chemicals were from Sigma

### A₃Membrane Preparation

### Buffers

Buffer A 10 mM HEPES, 0.9% NaCl, 0.2% EDTA, pH 7.4
Buffer B 10 mM HEPES, 10 mM EDTA, pH 7.4
Buffer C 10 mM HEPES, 0.1 mM EDTA, pH 7.4
A₃ culture media: 500 mL Iscoves Modified DMEM with Glutamax (Cat# 31980-022, Invitogen), 50 mL FCS (heat inactivated) (cat#10108-157, Invitrogen), 5 mL HEPES (1 M) (Cat# 15630-056, Invitrogen).

### Preparation Protocol

A3 CHO cells were cultured in roller bottles until 95% confluent at 37° C. and 5% CO₂.
40 mL ice-cold buffer A (lifting buffer) was then added and the roller bottle returned to the incubator for 10 minutes.
Cells were then scraped from the surface of the bottle using sterile scraper and transferred to a 50 mL Falcon tube on ice.
The surface of the roller was then washed with 10 mL of buffer A. This was transferred to the Falcon tube, which was then centrifuged at 500 g for 5 minutes at 4° C.
The supernatant was removed and 25 mL of ice-cold buffer B (lysis buffer) was added to the pellet.
The pellet was homogenized on ice using polytron (4 bursts of 5 seconds, with a 20-second interval separating each burst).
After homogenizing, the tubes were centrifuged 39,000×g for 25 minutes at 4° C. using a Beckman Avanti J-251 Ultracentrifuge.
The supernatant was removed and 20 mL of ice-cold buffer C (freezing buffer) was added to the tube.
The pellet was once again homogenized on ice using a polytron and then centrifuged at 39,000 g for 25 minutes at 4° C. on the Beckman Avanti J-251 Ultracentrifuge.
The supernatant was removed and the pellet was re-suspended in 1 mL of ice-cold buffer.
Protein quantification was estimated by the Bradford Protein Micro-Assay (BioRad®) using bovine serum albumin as a standard.
The membrane concentration was adjusted, aliquoted as required using buffer C and snap frozen prior to storage at −80° C.

### Binding Assay

### Buffers

Assay Buffer. 50 mM Tris-HCl, pH 7.4, 10 mM MgCl₂, 1 mM EDTA and 0.1% w/v BSA. Stored at 4° C. and kept for one week, once the BSA is added.
Wash Buffer. 50 mM Tris-HCl, pH 7.4 and 0.9% NaCl. Stored at 4° C.

### Compound Preparation

Ten (10) mM solutions of reference and test compounds were prepared in DMSO. The stock solutions were diluted in assay buffer containing 4% (v/v) DMSO to give a final concentration of 40 μM.

### K_{d} Determination

Radioligand binding to the CHO A₃ membranes was performed using radio-labelled agonist [¹²⁵I]-AB-MECA at a concentration range of 0.002-5 nM to obtain saturation binding. Binding experiments were performed in duplicate using 2.5 μg membrane in a total volume of 200 μL of assay buffer. The non-specific binding was determined in the presence of 10 μM of the agonist I-AB-MECA.

### Binding Assay

The assay was performed in a final volume of 200 μL/well, in a U-bottomed polypropylene 96-well plate. The components of the assay were added as follows:
50 μL test compound in assay buffer with 4% (v/v) DMSO. Total binding was determined using 50 μL vehicle. Non-specific binding was determined using 50 μL of 40 μM I-AB-MECA, to give a final assay concentration of 10 μM.
50 μL [¹²⁵I]-AB-MECA at a concentration of 1 nM (4×), to give a final assay concentration of 0.25 nM.
100 μL CHO A₃ membranes at a concentration of 25 μg/mL in assay buffer containing 4 U/mL adenosine deamin ase (ADA) (final assay concentration of 2 U/mL), to give a final assay concentration of 2.5 μg/well.

Compound dilution were prepared on a Biomek 2000 to give a series of 10 concentrations from 40-0.002 μM (4×). Fifty (50) μL of each concentration was transferred to a Dynex 96-well plate using a Tomtec Quadra. Total binding was determined in the absence of I-AB-MECA and non specific binding in the presence of 10 μM I-AB-MECA. The CHO A₃ membranes were thawed immediately prior to use and diluted to a concentration of 25 μg/mL in assay buffer containing adenosine deaminase at 4 U/mL (2×). The suspension was kept on ice until use. The radioligand [¹²⁵I]-AB-MECA was diluted and 50 μL added to all wells of the 96-well plate to give a final radioligand concentration of 0.25 nM. One hundred (100) μL of diluted membrane preparation was added to each well to give a total protein concentration of 2.5 μg/well and 50 μL of assay buffer was added per well. The 96-well plate was briefly mixed and incubated for 120 minutes at room temperature.

The samples from the assay plate were harvested onto the Unifilter GF/B plate (to which 50 μL of 0.5% (w/v) polyethyleneimine had been added to all the wells) using an automated Tomtec 9600 harvestor. The Unifilter GF/B plate was incubated for 3 hours at 50° C. or overnight at room temperature to dry the filters. Backing film was applied to the Unifilter GF/B plate, Microscint-20 was added to each well and the plate sealed using TopSeal-S according to the manufacturers instructions. The Unifilter GF/B plate was counted using a Packard TopCount (¹²⁵I-Scintillation, 1 min./well). The counts per minute (cpm) were used to determine IC₅₀ and from these a Ki was determined using the equation below. See Cheng and Prusoff, Biochem Pharmacol, Vol. 22, pp. 3099-3018 (1973).

$$Ki = \frac{{IC}_{50}}{1 + \frac{C}{Kd}} ,$$

where
C=concentration of radioligand; and
K_{d}=dissociation constant for the ligand.

### A₃ [³⁵S]-GTP_{Gamma}S Binding Functional Assay

### List of Abbreviations

| | |
|---|---|
| [³⁵S]-GTP?S | Guanosine 5′-[γ-³⁵S]thiotriphosphate, |
| | triethylammonium salt |
| BSA | Bovine serum albumin |
| CHO | Chinese hamster ovary |
| DMSO | Dimethyl sulphoxide |
| GDP | Guanosine 5′-diphosphate |
| GTP-?-S | Guanosine 5′-O-(3-thiotriphosphate) |
| HEPES | 4-(2-hydroxyethyl) piperazine-1-ethanesulfonic acid |
| I-AB-MECA | N6-(4-Amino-3-iodobenzyl)-5′-N-methylcarbamoyl- |
| | adenosine |
| MgCl₂ | Magnesium chloride |
| NaCl | Sodium chloride |
| SPA | Scintillation proximity assay |
| Tris-HCl | Tris(hydroxymethyl) aminomethane hydrochloride |
| WGA | Wheat germ agglutinin |

To establish the functional response to compounds of this invention an assay was carried out measuring A₃ agonist stimulation of [³⁵S]-GTPγS binding in membranes prepared from CHO cells stably expressing adenosine A₃ receptors. The agonist-induced stimulation of binding of [³⁵S]-GTPγS to activated G proteins has been used as a functional assay for a variety of receptors, including adenosine receptors. See Lorenzen et al., Mol Pharmacol, Vol. 49, pp. 915-926 (1996); and Jacobson et al., Drug Dev Res Vol. 37, p. 131 (1996).

A number of considerations must be taken into account when performing a [³⁵S]-GTPγS binding assay. Firstly, GDP is included in the assay to promote G-protein inactivation. Excess GDP may cause a decrease in catalytic rate of G-protein activation to which high efficacy agonists may be less susceptible. Low efficacy agonists may struggle to elicit a response where there are high concentrations of GDP. One possible theory why high efficacy agonists are able to overcome the GDP block is that they induce or stabilise changes in receptor conformation. Secondly, high concentrations of sodium ions are required to lower basal activity in the assay and as a result high affinity binding may be impaired. Thirdly, dissociation of the α- from the βγ subunit requires Mg²⁺ ions, which may effect the ability of certain agonists to bind. The presence of Mg²⁺ may also cause irreversible binding of GTPγS, thus a non-equilibrium state may occur. Finally, in [³⁵S]-GTPγS binding assays, GTPγS binds to all G-proteins, i.e., it does not distinguish between different G-proteins and as with other membrane protein assays, it is also susceptible to protein degradation by proteases.

The conventional GTPγS binding assay described by Lorenzen et al (1996), supra, is a filtration based method and thus requires a separation step; we have modified this method to run as a SPA format so that it can be used in a semi-automated and homogenous formal. In the SPA assay membranes are captured by wheatgerm agglutinin (WGA) SPA beads, through a specific interaction between WGA and carbohydrate residues of glycoproteins on the surfaces for the membranes. Upon receptor stimulation, [³⁵S]-GTPγS binds specifically to the alpha subunit of the G-protein thus bringing the [³⁵S]-GTPγS into close proximity with the SPA beads. Emitted β particles from the [³⁵S]-GTPγS excite the scintillant in the beads and produce light. Free [³⁵S]-GTPγS in solution is not in close proximity to the SPA beads and therefore does not activate the scintillant and hence does not produce light.

### Methods

### Materials

CHO adenosine A3 cells
N-2-Hydroxyethylpiperazine-N-2-thanesulfonic acid (HEPES) (Invitogen, Cat# 15630-056)
BSA (essentially fatty acid free) (Sigma, Cat# A-6003)
Tris (BDH Biochemicals, Cat# 443864E)
Ethylenediamine-tetra-acetic acid (EDTA) (Sigma, Cat# E-5391)
MgCl₂ (anhydrous) (Sigma, Cat# M-8266)
GDP (sodium salt) (Sigma, Cat# G-7127)
GTPγS (tetralithium salt) (Sigma, Cat# G-8634)
[³⁵S]-GTPγS (Amersham SJ1320, 1 μCi/μL)
WGA SPA beads (Amersham International, Cat# SPQ0031)
Polypropylene 96-well plates (Greiner, Cat# 650201)
White non-binding surface 96-well Optiplates: Packard Cat# 6005190
TopSeal—(Canberra Packard counter Cat# 6005185)

### A₃Membrane Preparation

### Buffers

Buffer A 10 mM HEPES, 0.9% NaCl, 0.2% EDTA, pH 7.4
Buffer B 10 mM HEPES, 10 mM EDTA, pH 7.4
Buffer C 10 mM HEPES, 0.1 mM EDTA, pH 7.4
A₃ culture media: 500 mL Iscoves Modified DMEM with Glutamax (Cat# 31980-022, Invitogen), 50 mL FCS (heat inactivated) (Cat# 10108-157, Invitrogen), 5 mL HEPES (1 M) (Cat# 15630-056, Invitrogen).

### Preparation Protocol

A₃ CHO cells were cultured in roller bottles until 95% confluent at 37° C. and 5% CO₂.
Forty (40) mL ice-cold buffer A (lifting buffer) was then added and the roller bottle returned to the incubator for 10 minutes.
Cells were then scraped from the surface of the bottle using sterile scraper and transferred to a 50 mL Falcon tube on ice.

The surface of the roller was then washed with 10 mL of buffer A. This was transferred to the Falcon tube, which was then centrifuged at 500 g for 5 minutes at 4° C.
The supernatant was removed and 25 mL of ice-cold buffer B (lysis buffer) was added to the pellet.
The pellet was homogenized on ice using polytron (4 bursts of 5 seconds, with a 20-second interval separating each burst).
After homogenizing, the tubes were centrifuged 39,000×g for 25 minutes at 4° C. using a Beckman Avanti J-251 Ultracentrifuge.
The supernatant was removed and 20 mL of ice-cold buffer C (freezing buffer) was added to the tube.
The pellet was once again homogenized on ice using a polytron and then centrifuged at 39,000 g for 25 minutes at 4° C. on the Beckman Avanti J-251 Ultracentrifuge.
The supernatant was removed and the pellet was re-suspended in 1 mL of ice-cold buffer.
Protein quantification was estimated by the Bradford Protein Micro-Assay (BioRad®) using bovine serum albumin as a standard.
The membrane concentration was adjusted, aliquoted as required using buffer C and snap frozen prior to storage at −80° C.

### Bead Storage Buffer

50 mM Tris-HCl (7.88 mg/mL), pH 7.4.
Solution was stored at 4° C.

### Assay Buffer

20 mM HEPES (4.766 g/L)
10 mM MgCl₂ (2.033 g/L)
100 mM NaCl (5.844 g/L)
1 mM EDTA (0.452 g/L)
pH 7.4
% BSA (1 g/L)

WGA PVT SPA beads were made to 250 mg/mL in assay buffer and stored at 4° C. for a maximum of one week.

[³⁵S]-GTPγS—concentration of the stock [³⁵S]-GTPγS was determined on the day in the following way:

$$The molarity ( μM ) of [ {}^{35} S ] \text{-} GTP γ S = \frac{radioactive concentration ( mCi \text{/} mL )}{specific activity of the stock ( Ci \text{/} mmol )} \times 1000$$

Example: At day 5, the activity is 0.961 μCi/μL (obtained from the table for radioactive decay of [³⁵S] at back of Amersham catalogue, reference=1 μCi/μL) therefore for a batch of [³⁵S]-GTPγS with specific activity 1082 Ci/mmol:

$$mCi \text{/} m mol , the molarity is \frac{0.961 \times 1000}{1082} = 0.888 μM$$

### Assay Protocol

The assay was performed in a final volume of 250 μL/well in a white non-binding surface 96-well Optiplate. Assay components were added as follows:
25 μL of assay buffer was added to all wells of 96-well Optiplate.
25 μL of 10 μM GDP was also added to each well.
To wells A1 to D1, and E12 to H12 add 25 μL of 10% DMSO/assay buffer—control to determine basal response.
To wells E1 to H1 and A12 to D12 add 25 μL of 100 nM 1-AB-MECA in 10% DMSO/assay buffer—control to determine maximal stimulation.
Compounds were diluted on Biomek with tip change (1 in 3 dilutions, in 10% DMSO/assay buffer), and 25 μL transferred in duplicate to Optiplates.
[³⁵S]-GTPγS was diluted to 1.25 nM (see above) and 25 μL added to each well to give a final assay concentration of 0.125 nM [³⁵S]-GTPγS/well.
Membranes were diluted in assay buffer to 25 μg/mL
The stock solution of SPA beads was diluted in assay buffer to give a concentration of 5 mg/mL.
Just prior to addition to the plate (no more than 20 minutes before use) the beads were mixed with the membranes 1:2 ratio (50 μL beads:100 μL of membrane).
One hundred fifty (150) μL of the beads and membrane mixture was added to each well.
The plate was sealed with TopSeal and incubated at room temperature for between 40 and 170 minutes.
The plate was centrifuged at 850×g for 10 minutes, at room temperature (Jouan B4i) and immediately read on Packard Topcount, program [³⁵S dpm] for 1 min./well.

Accordingly, agents of the invention can be useful for the treatment of a condition mediated by activation of the adenosine A₃ receptor.

For instance, The present invention can used to treat rheumatoid arthritis as described WO 04/045627.

Also, the present invention is based on the surprising finding that administration of A₃ adenosine receptor agonist (A₃RAg) alleviates symptoms of multiple sclerosis as described in WO 05/063246.

The present invention concerns, by one embodiment, a method for the treatment of multiple sclerosis (MS) in a human subject, comprising administering to an individual in need of such treatment an effective amount of an A₃RAg.

The term “multiple sclerosis” (MS) refers in the context of the present invention to the inflammatory disease of the CNS in which the nerve insulating myelin sheath is partially lost, resulting in various pathological symptoms. MS includes various types of the disease such as relapsing/remitting (RRMS), secondary progressive (SPMS), progressive relapsing (PRMS) and primary progressive (PPMS).

The terms “treatment” or “neuralgic protection” in the context of the present invention refer to any improvement in the clinical symptoms of the disease, and/or a reduction in the rate of deterioration or the relapse rate of the MS patient, as well as any improvement in the well being of the patients. For example, an improvement may be manifested by one or more of the following: decrease in muscle weakness, decrease in muscle spasms, reduction of spasticity, improvement of balance and improvement in memory.

The present invention is also based upon the finding that adenosine receptor agonists inhibit viral replication inside cells as described in WO 02/055085. Thus, in accordance with the invention, there is provided a method for inhibiting viral replication in cells, comprising presenting to the cells an effective amount of at least one A₃RAg.

The agonist according to the invention is either a full or partial agonist of the adenosine A₃ receptor. As used herein, a compound is a “full agonist” of an adenosine A₃ receptor if it is able to fully inhibit adenylate cyclase (A₃), a compound is a “partial agonist” of an adenosine A₃ receptor if it is able to partially inhibit adenylate cyclase (A₃).

Also provided by the invention are pharmaceutical compositions for inhibiting viral replication inside cells, comprising an effective amount of said at least one A₃RAg, as well as the use of said active ingredient (i.e., the A₃RAg) for the manufacture of such a pharmaceutical composition.

The invention is particularly useful, although not limited to, inhibiting the replication of HIV virus in human cells.

The method of the present invention can have particular usefulness in in vivo Applications as described in WO 95/02604. For example, as described in WO 95/02604, A₃ adenosine receptor agonists can be used in the treatment of any disease state or condition involving the release of inositol-1,4,5-triphosphate (IP3), diacylglycerol (DAG) and free radicals and subsequent arachidonic acid cascades. Thus, high blood pressure, locomotor hyperactivity, hypertension, acute hypoxia, depression, and infertility can be treated in accordance with the present inventive method, wherein one of the above-described compounds is acutely administered, e.g., within about a few minutes to about an hour of the onset or realization of symptoms. The method also has utility in the treatment of chronic disease states and conditions, in particular, those conditions and disease states wherein chronic prophylactic or therapeutic administration of one of the above-described compounds will prevent the onset of symptoms or will reduce recovery time. Examples of disease states and conditions that may be chronically treated in accordance with the present inventive method include inflammatory disorders, such as vascular inflammation and arthritis, allergies, asthma, wound healing, stroke, cardiac failure, acute spinal cord injury, acute head injury or trauma, seizure, neonatal hypoxia (cerebral palsy; prophylactic treatment involves chronic exposure through placental circulation), chronic hypoxia due to arteriovenous malformations and occlusive cerebral artery disease, severe neurological disorders related to excitotoxicity, Parkinson's disease, Huntington's chorea, and other diseases of the central nervous system (CNS), cardiac disease, kidney disease and contraception.

Moreover, the above compounds have been found to increase basal or systemic blood pressure, and thus the chronic administration of these compounds can be used to treat malignant hypotension. For example, the administration of IB-MECA results in a significant increase (e.g., about 10-30 t) in basal or systemic blood pressure (e.g., from about 70 mmHg to about 90 mmHg).

Such compounds have also been found to be significant cerebral protectants. As such, the above compounds can be used to treat and/or protect against a variety of disorders, including, e.g., seizures, transient ischemic shock, strokes, focal ischemia originating from thrombus or cerebral hemorrhage, global ischemia originating from cardiac arrest, trauma, neonatal palsy, hypovolemic shock, bronchiectasis, as agents for promoting sleep, as agents for treating demyelinating diseases, eg multiple sclerosis and as neuroprotective agents for eg, cerebral haemorrhagic injury, spinal cord ischaemi-reperfusion injury, hyperglycemia and associated neuropathies. The above compounds, particularly, e.g., IB-MECA, have also been found to have precognitive effects and, therefore, can be used in the treatment of disorders wherein the elicitation of such an effect would prove useful, such as in the treatment of Alzheimer's disease and other dementing and cognitive disorders.

According to WO 06/011130, administration of an A₃RAg to a human subject alleviated symptoms of Sjogren's syndrome (SS).

Thus, the present invention concerns, by one embodiment, a method for the treatment of SS in a human subject, comprising administering to an individual in need of such treatment an effective amount of an A₃RAg. In one preferred embodiment, the A₃Rag is administered topically, e.g., to the eye or skin. In another preferred embodiment, the A₃Rag is administered orally.

The term “SS” refers in the context of the present invention to the autoimmune disorder that causes KCS, in which immune cells attack and destroy the glands that produce tears and saliva. In one embodiment of the invention, the term refers to the disorder classified as secondary SS. In a preferred embodiment, the secondary SS results from a rheumatic condition. Symptoms of the disorder may include eye, mouth, skin, nose and vaginal dryness, and may affect other organs of the body including the kidneys, blood vessels, lungs, liver, pancreas and brain.

The method of the invention is contemplated as treating or preventing the opthalmologic clinical symptom and sign in dry eye including SS. The opthalmologic clinical symptom in SS includes but is not limited to foreign body sensation, burning and itching; and the opthalmologic clinical sign in SS includes but is not limited to, corneal and conjunctival erosions stained by fluorescein and rose Bengal, and tear film break-up time.

Agents of the invention can be used in combination with other active agents described in WO 01/23399, WO 95/02604, WO 05/063246, WO 02/055085 and WO 06/011130.

The agents of the invention may be administered by any appropriate route, e.g., orally, e.g., in the form of a tablet or capsule; parenterally, e.g., intravenously; by inhalation, or as described in WO 01/23399, WO 95/02604, WO 05/063246, WO 02/055085 and WO 06/011130.

In a further aspect, the invention also provides a pharmaceutical composition comprising a compound of formula (I), in free form or in the form of a pharmaceutically acceptable salt, optionally together with a pharmaceutically acceptable diluent or carrier therefor. The composition may contain a co-therapeutic agent, such as an anti-inflammatory, bronchodilatory, anti-histamine or anti-tussive drug, as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g., patches. Compositions for inhalation may comprise aerosol or other atomizable formulations or dry powder formulations. Other formulations can be as described in WO 01/23399, WO 9 5/02604, WO 05/063246, WO 02/055085 and WO 06/011130.

Dosages of compounds of formula (I) employed in practising the present invention will of course vary depending, e.g., on the particular condition to be treated, the effect desired and the mode of administration as described in WO 01/23399, WO 95/02604, WO 05/063246, WO 02/055085 and WO 06/011130.

The invention is illustrated by the following Examples.

### EXAMPLES 1-5

Compounds of formula I

| | |
|---|---|
| | I |
| 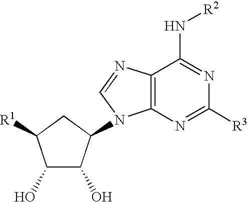 | |

| Ex. | R¹ | R² | R³ |
|---|---|---|---|
| 1 | 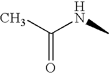 | 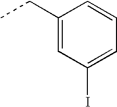 | Cl |
| 2 | 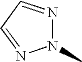 | 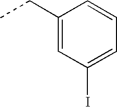 | Cl |
| 3 | 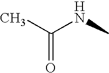 | CH₃ | 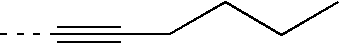 |
| 4 | 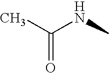 | 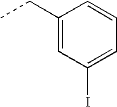 | H |
| 5 | 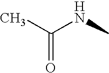 | 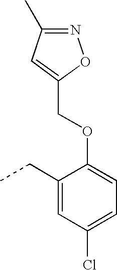 | H |

### Example 1

### (1S,4R)-4-(2,6 Dichloro-purin-9-yl)-cyclopent-2-enol

2,6-Dichloropurine (10 g, 52.90 mmol), (1S,4R)-cis 4-acetoxy-2-cyclopenten-1-ol (10 g, 70.40 mmol), tris(dibenzylideneacetone)dipalladium(0) (3.20 g, 3.50 mmol) and polymer supported triphenylphosphine (3 mmol/g, 11.60 g, 35.00 mmol) are placed in an oven-dried flask under an atmosphere of argon. Dry deoxygenated THF (80 L) is added and the reaction mixture is stirred gently for 5 minutes. Triethylamine (20 mL) is added and the reaction mixture is stirred at 50° C. The reaction is shown to be complete by LCMS after 1 hour. The reaction mixture is allowed to cool, filtered and the solvent is removed in vacuo. The title compound is obtained after purification by flash column chromatography (silica, dichloro methane:methanol 25:1).

¹H nmr (CDCl₃, 400 MHz); 8.30 (s, 1H), 6.40 (m, 1H), 5.90 (m, 1H), 5.50 (m, 1H), 4.95 (m, 1H), 3.05 (m, 1H), 2.10 (m, 1H), MS (ES+) m/e 271 (MH⁺).

### Carbonic Acid (1S,4R)-4-(2,6-dichloro-purin-9-yl)-cyclopent-2-enyl ester ethyl ester

(1S,4R)-4-(2,6-Dichloro-purin-9-yl)-cyclopent-2-enol (9.5 g, 35.05 mmol) is placed in an oven-dried flask under an atmosphere of argon. Dry THF (200 mL) is added followed by dry pyridine (5.54 g, 70.1 mmol). Ethyl chloroformate (15.21 g, 140.2 mmol) is added slowly so that the temperature does not rise above 40° C. and the reaction mixture is stirred at room temperature. The reaction is shown to be complete by LCMS after 1 hour. The solvent is removed in vacuo and the residue is partitioned between dichloromethane (200 mL) and water (200 mL). The organic layer is washed with water (150 mL) and brine (150 mL), dried over MgSO₄, filtered and the solvent is removed in vacuo. The title compound is obtained after crystallization from methanol.

¹H NMR (CDCl₃, 400 MHz); 8.20 (s, 1H), 6.45 (m, 1H), 6.25 (m, 1H), 5.75 (m, 1H), 5.70 (m, 1H), 4.25 (q, 2H), 3.20 (m, 1H), 2.05 (m, 1H), 1.35 (t, 3H), MS (ES+) m/e 343 (MH⁺).

### Di-Boc-[(1S,4R)-4-(2,6-dichloro-purin-9-yl)-cyclopent-2-enyl]-amine

Carbonic acid (1S,4R)-4-(2,6-dichloro-purin-9-yl)-cyclopent-2-enyl ester ethyl ester (2.5 g, 7.29 mmol), di-t-butyl iminodicarboxylate (1.74 g, 8.02 mmol), tris(dibenzylideneacetone)dipalladium(0) (0.33 g, 0.36 mmol) and triphenylphosphine (0.29 g, 1.09 mmol) are placed in an oven-dried flask under an atmosphere of argon. Dry deoxygenated THF (30 mL) is added and the reaction mixture is stirred at room temperature. The reaction is shown to be complete by LCMS after 3 hours. The solvent is removed in vacuo and the title compound is obtained after purification by flash column chromatography (silica, ethyl acetate:isohexane 4:1).

¹H NMR (CDCl₃, 400 MHz); 8.70 (s, 1H), 6.20 (m, 1H), 5.85 (m, 1H), 5.80 (m, 1H), 5.40 (m, 1H), 3.20 (m, 1H), 2.15 (m, 1H), 1.55 (s, 18H), MS (ES+) m/e 470 (MH⁺).

### (1S,2R,3S,5R)-3-(Di-tert-butoxycarbonylamino)-5-(2,6-dichloro-purin 9-yl)-cyclopentane-1,2-diol

A deep red/orange aqueous solution of ruthenium tetroxide was prepared by dissolving ruthenium trichloride trihydrate (60 mg, 0.29 mmol) in water (5 mL) with sodium periodate (682 mg, 3.19 mmol), and added in one portion to a chilled solution (ice/water bath to 0° C.) of (1S,4R)-1-(di-tert-butoxycarbonylamino)-4-(2,6-dichloropurin-9-yl)-cyclopent-2-ene (1.00 g, 2.12 mmol)) in ethyl acetate:acetonitrile 1:1 (30 mL). The resulting cloudy brown mixture was stirred on ice/water for 10 minutes, then quenched by the addition of saturated aqueous sodium metabisulfite (25 mL) and stirred for 1 hour. The mixture was diluted by the addition of ethyl acetate (75 mL), and washed consecutively with water (2×25 mL) and brine (20 mL), before drying over magnesium sulphate. Filtration and the removal of volatile components under reduced pressure gave the desired product as a pale yellow solid, which was used without further purification.

### (1S,2R,3S,5R)-3-(Di-tert-butoxycarbonylamino)-5-[2-chloro-6-(3-iodobenzylamino)-purin-9-yl]-cyclopentane-1,2-diol

3-Iodobenzylamine (500 mg, 2.15 mmol) and triethylamine (400 μL, 291 mg, 2.9 mmol) were dissolved in dichloromethane (5 mL) and added to a solution of (1S,2R,3S,5R)-3-(di-tert-butoxycarbonylamino)-5-(2,6-dichloro-purin-9-yl)-cyclopentane-1,2-diol (1.07 g, 2.12 mmol) in dichloromethane (20 mL). The reaction was stirred at ambient temperature for 4 days, before removing the volatile components under reduced pressure. The desired product was purified from the crude residue by flash column chromatography, using the Argonaut Flashmaster Personal system. The residue was loaded in the minimum amount of dichloromethane onto a 70 g Varian Megabond Elut Flash Si cartridge, presaturated with isohexane. The product was purified by elution with isohexane (250 mL), followed by 1:1 ethyl acetate isohexane (1 L); the pure fractions were combined and the solvent removed under reduced pressure to give the product as a beige foam (610 mg; 41% yield). LC-MS: MH⁺ 701.49.

### (1S,2R,3S,5R)-3-Amino-5-[2-chloro-6-(3-iodo-benzylamino)-purin-9-yl]-cyclopentane-1,2-diol

(1S,2R,3S,5R)-3-(Di-tert-butoxycarbonylamino)-5-[2-chloro-6-(3-iodobenzylamino)-purin-9-yl]-cyclopentane-1,2-diol (590 mg, 0.84 mmol) was dissolved in methanol (10 mL); 4.0 M hydrogen chloride in 1,4-dioxane (10 mL) was added, and the pale yellow solution was stirred at ambient temperature for 1 hour, after which time the reaction was seen to be complete by TLC. The volatile components were removed under reduced pressure, to give a beige solid (450 mg, quantitative yield). LC-MS: MH⁺ 501.15.

### N-{(1S,2R,3S,4R)-4-[2-Chloro-6-(3-iodobenzylamino)-purin-9-yl]-2,3-dihydroxycyclopentyl}-acetamide

(1S,2R,3S,5R)-3-Amino-5-[2-chloro-6-(3-iodo-benzylamino)-purin-9-yl]-cyclopentane-1,2-diol (450 mg, 0.84 mmol) was suspended in dichloromethane (10 mL) with triethylamine (380 μL, 275 mg, 2.73 mmol). Acetyl chloride (65 μL, 72 mg, 0.91 mmol) was added, and the resulting pale yellow solution was stirred at ambient temperature for 1 hour. Methanol (5 mL) was added to quench any residual acetyl chloride, and all volatile components were removed under reduced pressure, to give a brown foam. The product was initially purified by flash column chromatography, using the Argonaut Flashmaster Personal system. The brown foam was dissolved in dichloromethane (10 mL) and adsorbed onto silica (3 g). This was loaded onto a 20 g Isolute Flash Si cartridge, presaturated with ethyl acetate. The product was eluted with 5% methanol in ethyl acetate, and removal of the solvent from the fractions containing the purified product under reduced pressure gave a colorless solid. Crystallization from methanol gave a colorless crystalline solid (210 mg, 46% yield). LC-MS: MH⁺ 543.17

### Example 2

### 2,6-Dichloro-9-((1R,4S)-4-[1,2,3]triazol-2-yl-cyclopent-2-enyl)-9H-purine

Carbonic acid (1S,4R)-4-(2,6-dichloro-purin-9-yl)-cyclopent-2-enyl ester ethyl ester (1.0 g, 2.91 mmol) was dissolved in dry deoxygenated THF (20 mL) under argon. Triphenyl phosphine (115 mg, 0.44 mmol, 0.15 equivalents), [1,2,3]triazole (200 μL, 238 mg, 3.45 mmol) and Pd₂(dba)₃ (133 mg, 0.146 mmol, 5 mol %) were added sequentially. The reaction mixture was stirred at 50° C. for 2 hours, and allowed to cool to room temperature, before the volatile components were removed under reduced pressure. The product was purified by flash column chromatography, using the Argonaut Flashmaster Personal. The residue was re-suspended in dichloromethane (5 mL) before loading onto a 25 g Isolute Flash Si cartridge, presaturated with isohexane. The product was eluted after isohexane (500 mL), isohexane ethyl acetate 4:1 (250 mL) and isohexane:ethyl acetate 1:1 (750 mL). The solvent was removed from the fractions containing pure product under reduced pressure, and the product was re-crystallized from ethyl acetate, to give a beige solid (280 mg, 30% yield). LC-MS MH⁺ 321.80

### (1R,2S,3R,5S)-3-(2,6-Dichloro-purin-9-yl)-5-[1,2,3]triazol-2-yl-cyclopentane-1,2-diol

2,6-Dichloro-9-((1R,4S)-4-[1,2,3]triazol-2-yl-cyclopent-2-enyl)-9H-purine (1 equivalent) was dissolved in THF (0.1 M) with N-methylmorpholine-N-oxide (2 equivalents). Osmium tetroxide was added as a 4% solution in water (10 mol %), and the reaction was stirred at room temperature for 24 hours, before a further addition of 4% OSO_{4(aq)} (10 mol %) and stirring for another 24 hours. The reaction was diluted with ethyl acetate, and washed with 0.2 M HCl_{(aq)}, then brine, before drying over magnesium sulfate. Filtration and removal of the solvent under reduced pressure gave the crude product, to be purified by flash column chromatography/crystallization.

### (1R,2S,3R,5S)-3-[2-Chloro-6-(3-iodo-benzylamino)-purin-9-yl]-5-[1,2,3]triazol-2-yl-cyclopentane-1,2-diol

3-Iodobenzylamine (1 equivalent) and triethylamine (1.1 equivalents) were dissolved in dichloromethane (˜0.4 M w.r.t. 3-iodobenzylamine) and added to a solution of (1R,2S,3R,5S)-3-(2,6-dichloropurin-9-yl)-5-[1,2,3]triazol-2-yl-cyclopentane-1,2-diol in dichloromethane (1 equivalent; 0.1 M). The reaction was stirred at room temperature overnight, before removal of the volatile components under reduced pressure. The desired product was purified by flash column chromatography/crystallization.

### Example 3

### (1S,4R)-4-(6-Chloro-2-iodo-purin-9-yl)-cyclopent-2-enol

6-Chloro-2-iodo-purine (see Taddei et al., Org Biomol Chem, Vol. 2, pp. 665-670 (2004); 1 equivalent), (1S,4R)-cis-4-acetoxy-cyclopent-2-enol (1.33 equivalents) and polymer bound triphenyl phosphine (0.66 equivalents) were combined and placed under vacuum at room temperature for 24 hours. Freshly distilled, deoxygenated THF was added (to 1.0 M w.r.t the (1S,4R)-cis-4-acetoxy-cyclopent-2-enol), followed by Pd₂(dba)₃ (5 mol %). The mixture was stirred for 15 minutes at room temperature, before triethylamine (dried over potassium hydroxide) was added (3 equivalents). The reaction mixture was stirred for 1 hour at 50° C., allowed to cool to room temperature and filtered. The volatile components were removed under reduced pressure, and the product purified by flash column chromatography/crystallization.

### Carbonic Acid (1S,4R)-4-(6-chloro-2-iodo-purin-9-yl)-cyclopent-2-enyl ester ethyl ester

Pyridine (3 equivalents) was added to a 0.2 M solution of (1S,4R)-4-(6-chloro-2-iodo-purin-9-yl)-cyclopent-2-enol (1 equivalent) in dry THF. Ethyl chloroformate (4 equivalents) was slowly added, ensuring the reaction temperature did not rise above 40° C. Once addition was complete, the reaction was stirred at room temperature until complete. Any precipitate was removed by filtration, and the volatile components were removed under reduced pressure. The residue was taken up in dichloromethane, and washed consecutively with 0.1 M hydrochloric acid, water (×2) and brine, before drying over magnesium sulfate. Filtration and removal of solvent under reduced pressure, followed by purification by flash column chromatography/crystallization, gave the desired product.

### Acetyl-[(1S,4R)-4-(6-chloro-2-iodo-purin-9-yl)-cyclopent-2-enyl]-carbamic acid tert-butyl ester

Carbonic acid (1S,4R)-4-(6-chloro-2-iodo-purin-9-yl)-cyclopent-2-enyl ester ethyl ester (1 equivalent), acetyl-carbamic acid tert-butyl ester (see Tanaka et al., Chem Pharm Bull, Vol. 36, No. 8, pp. 3215-3129 (1988); 1.15 equivalents) and triphenyl phosphine (0.15 equivalents) were combined in an oven-dried flask under an atmosphere of argon. Dry deoxygenated THF (to 0.3 M w.r.t. carbonic acid (1S,4R)-4-(6-chloro-purin-9-yl)-cyclopent-2-enyl ester ethyl ester) was added, followed by Pd₂(dba)₃ (5 mol %). The reaction mixture was stirred at 50° C. for 1 hour, and allowed to cool to room temperature, before the volatile components were removed under reduced pressure and the product purified by flash column chromatography/crystallization.

### Acetyl-[(1S,2R,3S,4R)-4-(6-chloro-2-iodo-purin-9-yl)-2,3-dihydroxy-cyclopentyl]-carbamic acid tertbutyl ester

Acetyl-[(1S,4R)-4-(6-chloro-purin-9-yl)-cyclopent-2-enyl]-carbamic acid tert-butyl ester (1 equivalent), methanesulfonamide (1 equivalent) and AD-mix-α (1.5 g/mmol substrate) were combined in tert-butanol:water 1:1 (to 0.1 M w.r.t. acetyl-[(1S,4R)-4-(6-chloro-purin-9-yl)-cyclopent-2-enyl]-carbamic acid tert-butyl ester). Osmium tetroxide (5 mol %, as a 4% solution in water) was added, and the reaction mixture was stirred vigorously overnight. Once complete, the reaction was partitioned between ethyl acetate and water; the organic phase was washed consecutively with fresh water (×2) and brine, before drying over magnesium sulfate. Filtration and removal of the volatile components under reduced pressure gave the desired product.

### Acetyl-[(1S,2R,3S,4R)-2,3-dihydroxy-4-(2-iodo-6-methylamino-purin-9-yl)-cyclopentyl]-carbamic acid tert-butyl ester

Acetyl-[(1S,2R,3S,4R)-4-(6-chloro-2-iodo-purin-9-yl)-2,3-dihydroxy-cyclopentyl]-carbamic acid tert-butyl ester was added to a large excess of liquid methylamine at −20° C., and stirred for 30 minutes, before allowing to warm the room temperature. The desired product was purified by flash column chromatography/crystallization.

### N-[(1S,2R,3S,4R)-2,3-Dihydroxy-4-(2-iodo-6-methylamino-purin-9-yl)-cyclopentyl]-acetamide

Acetyl-[(1S,2R,3S,4R)-2,3-dihydroxy-4-(2-iodo-6-methylamino-purin-9-yl)-cyclopentyl]-carbamic acid tert-butyl ester was dissolved in dichloromethane (˜0.1 M) and chilled on ice/water to 0° C. Sufficient trifluoroacetic acid was added to give a 20% solution, and the reaction was stirred on ice until complete. The volatiles were removed under reduced pressure, and the product purified by flash column chromatography/crystallization.

### N-[(1S,2R,3S,4R)-4-(2-Hex-1-ynyl-6-methylamino-purin-9-yl-2,3-dihydroxy-cyclopentyl]-acetamide

A 0.05 M solution of N-[(1S,2R,3S,4R)-2,3-dihydroxy-4-(2-iodo-6-methylamino-purin-9-yl)-cyclopentyl]-acetamide (1 equivalent) in a 7:2 mixture of dry DMF and triethylamine was prepared. To this was added copper (I) iodide (1 equivalent) and bis(triphenylphosphine)palladium dichloride (2 mol %), followed by 1-hexyne (6 equivalents). The resulting mixture was stirred at room temperature until complete and the volatile components were removed under reduced pressure. The product was purified by flash column chromatography/crystallization.

### Example 4

### (1S,4R)-4-(6-Chloro-purin-9-yl)-cyclopent-2-enol

6-Chloropurine (1 equivalent), (1S,4R)-cis-4-acetoxy-cyclopent-2-enol (1.33 equivalents) and polymer bound triphenyl phosphine (0.66 equivalents) were combined and placed under vacuum at room temperature for 24 hours. Freshly distilled, deoxygenated THF was added (to 1.0 M with respect to the (1S,4R)-cis-4-acetoxy-cyclopent-2-enol), followed by Pd₂(dba)₃ (5 mol %). The mixture was stirred for 15 minutes at room temperature, before triethylamine (dried over potassium hydroxide) was added (3 equivalents). The reaction mixture was stirred for one hour at 50° C., allowed to cool to room temperature and filtered. The volatile components were removed under reduced pressure, and the product purified by flash column chromatography/crystallization.

### Carbonic Acid (1S,4R)-4-(6-chloro-purin-9-yl)-cyclopent-2-enyl ester ethyl ester

Pyridine (3 equivalents) was added to a 0.2 M solution of (1S,4R)-4-(6-chloro-purin-9-yl)-cyclopent-2-enol (1 equivalent) in dry THF. Ethyl chloroformate (4 equivalents) was slowly added, ensuring the reaction temperature did not rise above 40° C. Once addition was complete, the reaction was stirred at room temperature until complete. Any precipitate was removed by filtration, and the volatile components were removed under reduced pressure. The residue was taken up in dichloromethane, and washed consecutively with 0.1 M hydrochloric acid, water (×2) and brine, before drying over magnesium sulfate. Filtration and removal of solvent under reduced pressure, followed by purification by flash column chromatography/crystallization, gave the desired product.

### Acetyl-[(1S,4R)-4-(6-chloro-purin-9-yl)-cyclopent-2-enyl]-carbamic acid tertbutyl ester

Carbonic acid (1S,4R)-4-(6-chloro-purin-9-yl)-cyclopent-2-enyl ester ethyl ester (1 equivalent), acetyl-carbamic acid tert-butyl ester (see Tanaka et al. (1988), supra; 1.15 equivalents) and triphenyl phosphine (0.15 equivalents) were combined in an oven-dried flask under an atmosphere of argon. Dry deoxygenated THF (to 0.3 M with respect to carbonic acid (1S,4R)-4-(6-chloro-purin-9-yl)-cyclopent-2-enyl ester ethyl ester) was added, followed by Pd₂(dba)₃ (5 mol %). The reaction mixture was stirred at 50° C. for 1 hour, and allowed to cool to room temperature, before the volatile components were removed under reduced pressure and the product purified by flash column chromatography/crystallization.

### Acetyl-[(1S,2R,3S,4R)-4-(6-chloro-purin-9-yl)-2,3-dihydroxy-cyclopentyl]-carbamic acid tert-butyl ester

Acetyl-[(1S,4R)-4-(6-chloro-purin-9-yl)-cyclopent-2-enyl]-carbamic acid tert-butyl ester (1 equivalent), methanesulfonamide (1 equivalent) and AD-mix-α (1.5 g/mmol substrate) were combined in tert-butanol:water 1:1 (to 0.1 M with respect to acetyl-[(1S,4R)-4-(6-chloro-purin-9-yl)-cyclopent-2-enyl]-carbamic acid tert-butyl ester). Osmium tetroxide (5 mol %, as a 4% solution in water) was added, and the reaction mixture was stirred vigorously overnight. Once complete, the reaction was partitioned between ethyl acetate and water; the organic phase was washed consecutively with fresh water (×2) and brine, before drying over magnesium sulfate. Filtration and removal of the volatile components under reduced pressure gave the desired product.

### Acetyl-{(1S,2R,3S,4R)-2,3-dihydroxy-4-[6-(3-iodo-benzylamino)-purin-9-yl]-cyclopentyl}-carbamic acid tertbutyl ester

3-Iodobenzylamine (1 equivalent) and triethylamine (1.1 equivalents) were dissolved in dichloromethane (˜0.4 M with respect to 3-iodobenzylamine) and added to a solution of acetyl-[(1S,2R,3S,4R)-4-(6-chloro-purin-9-yl)-2,3-dihydroxycyclopentyl]-carbamic acid tert-butyl ester in dichloromethane (1 equivalent; 0.1 M). The reaction was stirred with heating overnight, before removing the volatile components under reduced pressure. The desired product was purified by flash column chromatography/crystallization.

### N-{(1S,2R,3S,4R)-2,3-Dihydroxy-4-[6-(3-iodo-benzylamino)-purin-9-yl]cyclopentyl}-acetamide

Acetyl-{(1S,2R,3S,4R)-2,3-dihydroxy-4-[6-(3-iodo-benzylamino)-purin-9-yl]-cyclopentyl}-carbamic acid tertbutyl ester was dissolved in dichloromethane (˜0.1 M) and chilled on ice/water to 0° C. Sufficient trifluoroacetic acid was added to give a 20% solution, and the reaction was stirred on ice until complete. The volatiles were removed under reduced pressure, and the product purified by flash column chromatography/crystallization.

### Example 5

### Acetyl-((1S,2R,3S,4R)-4-{6-[5-chloro-2-(3-methyl-isoxazol-5-ylmethoxy)-benzylamino]-purin-9-yl}-2,3-dihydroxy-cyclopentyl)-carbamic acid tert-butyl ester

5-Chloro-2-(3-methylisoxazol-5-ylmethoxy)benzylamine (see DeNinno, et al., J Med Chem, Vol. 46, pp. 353-355 (2003) supplementary material; 1 equivalent) and triethylamine (1.1 equivalents) were dissolved in dichloromethane (˜0.4 M with respect to 3-iodobenzylamine) and added to a solution of acetyl-[(1S,2R,3S,4R)-4-(6-chloro-purin-9-yl)-2,3-dihydroxycyclopentyl]-carbamic acid tert-butyl ester in dichloromethane (1 equivalent; 0.1 M). The reaction was stirred with heating overnight, before removing the volatile components under reduced pressure. The desired product was purified by flash column chromatography/crystallization.

### N-((1S,2R,3S,4R)-4-{6-[5-Chloro-2-(3-methyl-isoxazol-5-ylmethoxy)-benzylamino]purin-9-yl}2,3-dihydroxy-cyclopentyl)-acetamide

Acetyl-((1S,2R,3S,4R)-4-{6-[5-chloro-2-(3-methyl-isoxazol-5-ylmethoxy)-benzylamino]-purin-9-yl}-2,3-dihydroxy-cyclopentyl)-carbamic acid tert-butyl ester was dissolved in dichloromethane (˜0.1 M) and chilled on ice/water to 0° C. Sufficient trifluoroacetic acid was added to give a 20% solution, and the reaction was stirred on ice until complete. The volatiles were removed under reduced pressure, and the product purified by flash column chromatography/crystallization.

1. A compound of formula I 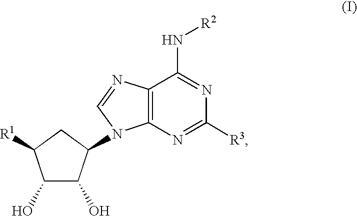
in free or salt form, wherein
R¹ denotes a N-bonded 3- to 12-membered heterocyclic group containing from 1 to 4 ring nitrogen atoms and optionally containing from 1 to 4 other heteroatoms selected from the group consisting of oxygen and sulfur, that group being optionally substituted by oxo, C₁-C₈-alkoxy, C₆-C₁₀-aryl, R^{1a} or by C₁-C₈-alkyl optionally substituted by OH, or
R¹ is —NH—C₁-C₈-alkylcarbonyl, —NH—C₃-C₈-cycloalkylcarbonyl, —NH—SO₂—C₁-C₈-alkyl, —NH—C₇-C₁₄-aralkylcarbonyl, —NH—C(═O)-3- to 12-membered heterocyclic group, —NH—C(═O)—C₆-C₁₀-aryl or —NH—C(═O)—C(═O)—NH—C₁-C₈-alkyl optionally substituted by R^{1a}, where R^{1a} is a 3- to 12-membered heterocyclic group containing at least one ring heteroatom selected from the group consisting of nitrogen, oxygen and sulphur, said 3- to 12-membered heterocyclic ring being optionally substituted by halo, cyano, oxo, OH, carboxy, amino, nitro, C₁-C₈-alkyl, C₁-C₈-alkylsulfonyl, aminocarbonyl, C₁-C₈-alkylcarbonyl or C₁-C₈-alkoxy optionally substituted by aminocarbonyl;
R² is selected from the group consisting of C₁-C₈-alkyl, R— and S— 1-phenylethyl, an unsubstituted benzyl group, and a phenylethyl or benzyl group substituted in one or more positions with a substituent selected from the group consisting of C₁-C₈-alkyl, amino, halo, C₁-C₈-haloalkyl, nitro, OH, acetamido, C₁-C₈-alkoxy and sulfo, or
R² is 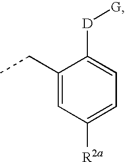
where
R^{2a} is halo, trifluoromethyl, cyano, C₁-C₈-alkyl, C₁-C₈-alkyloxy, ethenyl or ethynyl;
D is oxy, thio, NH, C₁-C₈-alkyloxy, C₁-C₈-alkylthio or —CO-alkylamino; and
G is a partially saturated, fully saturated or fully unsaturated 5- to 8-membered ring optionally having 1 to 3 heteroatoms selected independently from oxygen, sulfur and nitrogen, or, a bicyclic ring consisting of two fused partially saturated, fully saturated or fully unsaturated 3- to 6-membered rings, taken independently, optionally having 1 to 4 heteroatoms selected independently from nitrogen, sulfur and oxygen; wherein said G is optionally mono-, di- or tri-substituted independently with halo, C₁-C₈-alkyl, trifluoromethyl, trifluoromethoxy, nitro, cyano, C₃-C₁₀-cycloalkyl, hydroxy or C₁-C₈-alkoxy, or
G is cyano, C₁-C₈-alkoxycarbonyl, C₃-C₁₀-cycloalkoxycarbonyl, C(O)NR⁴R⁵, C(S)NR⁴R⁵, C(NH)NR⁴NR⁵, C(N(C₁-C₃)alkyl)NR⁴R⁵ or C(N(C₃-C₁₀)cycloalkyl)NR⁴R⁵;
R³ is selected from H, halo, C₁-C₈-alkyl optionally substituted by halo or OH, C₁-C₈-alkoxy, amino, C₁-C₈-alkylamino, C₂-C₁₀-alkenes, C₂-C₁₀-alkynes optionally substituted by C₁-C₈-alkyl, aryl optionally substituted by C₁-C₈-alkyl or OH, thio and C₁-C₈-alkylthio;
R⁴ is a bond, H, C₁-C₁₀-alkyl, hydroxy, C₁-C₁₀-alkoxy, C₃-C₁₀-cycloalkoxy or a partially saturated, fully saturated or fully unsaturated 5- to 8-membered ring, optionally linked through C₁-C₈-alkyl, optionally having 1 to 3 heteroatoms selected independently from oxygen, sulfur and nitrogen, or, a bicyclic ring or a bicyclic ring with optional C₁-C₈-bridge optionally linked through C₁-C₈-alkyl, said bicyclic ring or bridged bicyclic ring optionally having 1 to 4 heteroatoms selected independently from nitrogen, sulfur and oxygen wherein said C₁-C₁₀-alkyl, C₁-C₁₀-alkoxy, C₃-C₁₀-cycloalkoxy or R⁴ ring(s) is optionally mono-, di- or tri-substituted independently with halo, C₁-C₈-alkyl, trifluoromethyl, nitro, cyano, C₃-C₁₀-cycloalkyl, OH or C₁-C₈-alkoxy;
R⁵ is a bond, H, C₁-C₈-alkyl or C₁-C₁₀-cycloalkyl, and
R⁴ and R⁵, taken together with the nitrogen to which they are attached, form a fully saturated or partially unsaturated four to nine membered ring, said ring optionally bridged, optionally having 1 to 3 heteroatoms selected independently from oxygen, sulfur and nitrogen, said ring optionally mono- or di-substituted independently with oxo, hydroxy, C₁-C₈-alkoxy, C₁-C₈-alkyl, amino, mono-N— or di-N,N—C₁-C₈-alkylaminocarbonyl, mono-N— or di-N,N—C₃-C₁₀-cycloalkylaminocarbonyl, N—C₁-C₈-alkyl-N—C₃-C₁₀-cycloalkylaminocarbonyl, mono-N— or di-N,N—C₁-C₈-alkylamino, mono-N— or di-N,N—C₃-C₁₀-cycloalkylamin, N—C₁-C₈-alkyl-N—C₃-C₁₀-cycloalkylamino, formylamino, C₁-C₈-alkylcarbonylamino, C₃-C₁₀-cycloalkylcarbonylamino, C₁-C₈-alkoxycarbonylamino, N—C₁-C₈-alkoxycarbonyl-N—C₁-C₈-alkylamino, C₁-C₈-sulfamoyl, C₁-C₈-alkylsulfonylamino, C₃-C₁₀-cycloalkylsulfonylamino or a partially saturated, fully saturated or fully unsaturated 5- to 8-membered ring, optionally linked through C₁-C₈-alkyl, optionally having 1 to 3 heteroatoms selected independently from oxygen, sulfur and nitrogen, or, a bicyclic ring consisting of two fused partially saturated, fully saturated or fully unsaturated 3- to 6-membered rings, taken independently, optionally linked through C₁-C₈-alkyl, optionally having 1 to 4 heteroatoms selected independently from nitrogen, sulfur and oxygen, and optionally mono- or di-substituted with halo, trifluoromethyl, trifluoromethoxy, C₁-C₈-alkyl or C₁-C₈-alkoxy.

2. A compound according to claim 1,
wherein
R¹ denotes a N-bonded 3- to 12-membered heterocyclic group containing from 1 to 4 ring nitrogen atoms and optionally containing from 1 to 4 other heteroatoms selected from the group consisting of oxygen and sulphur, or
R¹ is —NH—C₁-C₈-alkylcarbonyl;
R² is C₁-C₈-alkyl or benzyl optionally substituted by halogen, or
R² is 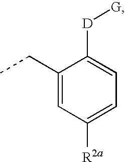
where
R^{2a} is halo, trifluoromethyl, cyano, C₁-C₈-alkyl, C₁-C₈-alkoxy, ethenyl or ethynyl;
D is oxy, thio, NH, C₁-C₈-alkoxy, C₁-C₈-alkylthio or —CO-alkylamino; and
G is a partially saturated, fully saturated or fully unsaturated 5- to 8-membered ring optionally having 1 to 3 heteroatoms selected independently from oxygen, sulfur and nitrogen, or, a bicyclic ring consisting of two fused partially saturated, fully saturated or fully unsaturated 3- to 6-membered rings, taken independently, optionally having 1 to 4 heteroatoms selected independently from nitrogen, sulfur and oxygen; wherein said G is optionally mono-, di- or tri-substituted independently with halo, C₁-C₈-alkyl; and
R³ is selected from H, halo, C₁-C₈-alkyl optionally substituted by halo or OH, C₁-C₈-alkoxy, amino, C₁-C₈-alkylamino, C₂-C₁₀-alkenes, C₂-C₁₀-alkynes optionally substituted by C₁-C₈-alkyl, C₆-C₁₀-aryl optionally substituted by C₁-C₈-alkyl or OH, thio and C₁-C₈-alkylthio.

3. A compound according to claim 1,
wherein
R¹ is a 5 to 12-membered heterocyclic group containing at least one ring heteroatom selected from the group consisting of nitrogen, oxygen and sulphur, or
R¹ is —NH—C₁-C₈-alkylcarbonyl;
R² is 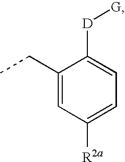
where
R^{2a} is halogen;
D is C₁-C₈-alkoxy; and
G is a 5-membered heterocyclic group substituted by a methyl group, or
R² is a benzyl substituted by halogen, or
R² is C₁-C₈-alkyl; and
R³ is H, halo or C₂-C₁₀-alkynes optionally substituted by C₁-C₈-alkyl.

4. A compound of formula I, according to claim 1 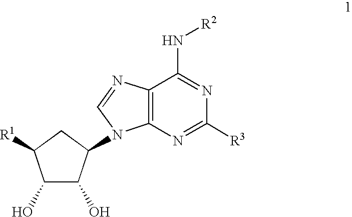
where R¹, R² and R³ are

| R¹ | R² | R³ |
|---|---|---|
| 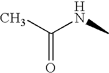 | 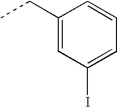 | Cl |
| 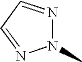 | 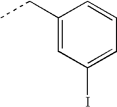 | Cl |
| 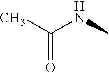 | CH₃ | 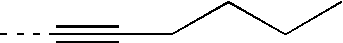 |
| 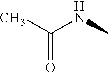 | 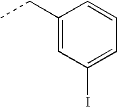 | H |
| 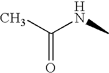 | 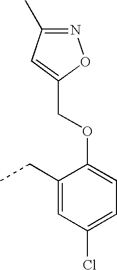 | H |

5. A compound according to claim 1 for use as a pharmaceutical.

6. Pharmaceutical compositions comprising a compound according to claim 1.

7. The use of a compound according to claim 1, in the manufacture of a medicament for the treatment of a condition mediated by activation of the adenosine A₃ receptor.

8. The use of a compound according to claim 7, wherein said condition mediated by activation of the adenosine A₃ receptor is rheumatoid arthritis.

9. A process for the preparation of compounds of formula (I) 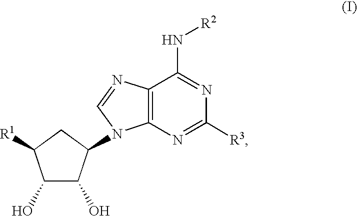
where R¹, R² and R³ are as defined hereinbefore, which comprises the steps of:
(i) (A) for the preparation of compounds of formula (I), reacting a compound of formula (Ia) 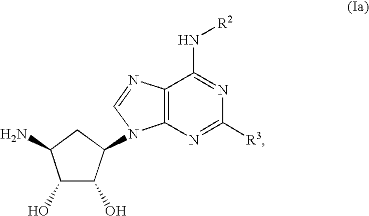
where R² and R³ are as hereinbefore defined, with acetyl chloride in the presence of base;
(B) for the preparation of compounds of formula (I), where R³ is C₂-C₈-alkynyl, reacting a compound of formula (Ib) 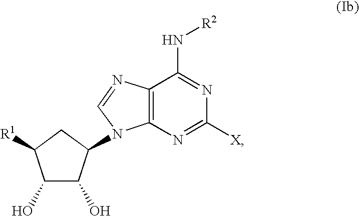
where X is a leaving group, with a compound of formula 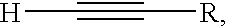
where R can be C₁-C₆-alkyl;
(C) for the preparation of compounds of formula (I), reacting a compound of formula (Ic) 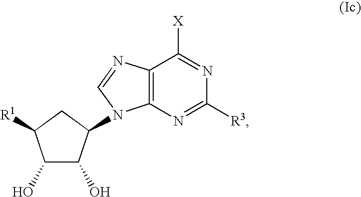
where
R¹ and R³ are as hereinbefore defined; and
X is a leaving group, with a compound of formula H₂N—R², where R² is as hereinbefore defined in the presence of a base; and
(ii) recovering the resultant compound of formula (I), in free or pharmaceutically acceptable salt form.
